(19) United States
(12) Patent Application Publication
(10) Pub. No.: US 2012/0244102 A1
(43) Pub. Date: Sep. 27, 2012

(54) AGENT FOR PREVENTING OR TREATING CROHN'S DISEASE, COMPRISING ORGANIC ACID POLYMER

(75) Inventors: Hiroyuki Hashimoto, Nagoya-shi (JP); Toshihiko Kumazawa, Nagoya-shi (JP); Motohiro Takeda, Nagoya-shi (JP); Takahito Jomori, Nagoya-shi (JP); Chihiro Hibi, Nagoya-shi (JP); Kuniharu Mizuno, Nagoya-shi (JP)

(73) Assignee: SANWA KAGAKU KENKYUSHO CO., LTD., Aichi (JP)

(21) Appl. No.: 13/514,083

(22) PCT Filed: Jan. 26, 2011
(86) PCT No.: PCT/JP2011/051425

§ 371 Date: Jun. 6, 2012

(30) Foreign Application Priority Data
Jan. 28, 2010 (JP) 2010-016166

(51) Int. Cl. A61K 31/765; A61P 1/04

(52) U.S. Cl. 424/78.01

(57) ABSTRACT

The present invention is to provide an agent for treating Crohn's disease that is of unknown cause and for which no radical therapeutic drug is present. Provided is an agent for preventing or treating Crohn's disease, comprising an organic acid polymer represented by the following formula: [(O_{1/2})₃Ge-A-CO₂H]ₙ, wherein n is 100-1,000, and A is a lower alkyl group. Preferably, A is a C1-C3 lower alkyl group and the degree of polymerization n is 200-900. The organic acid polymer according to the present invention is effective as an agent for treating Crohn's disease.

### TECHNICAL FIELD

The present invention relates to a novel pharmaceutical use of an organic acid polymer, specifically of a 3-oxygermylpropionic acid polymer.

### BACKGROUND ART

Crohn's disease (CD) is an intractable disease of unknown cause by which lesions such as stenosis or fistula appear incontinuously on the whole area of the gastrointestinal tract mainly from the oral cavity to the anus. Furthermore, in some cases, the disease is accompanied by extraintestinal complications such as arthritis, iritis, pyoderma gangrenosum and erythema nodosum. In Japan, the disease is designated as one of target diseases (specified diseases) in the Intractable Disease Treatment Research Program, and is an intractable disease that is different from gastric ulcer and duodenal ulcer. There is no radical therapeutic method, and the object of the therapy is to suppress the symptom in the active stage to induce remission, and to maintain the remission state by the medicinal therapy or the like. The bases of an oral pharmacotherapy are 5-aminosalicylic acid (5-ASA) formulations such as mesalazine and salazosulfapyridine, and adrenocortical steroids (prednisolone) in Japan, and in some cases, antibacterial agents (metronidazole), immunosuppressive agents (azathioprine) and the like is administered. On the other hand, adrenocortical steroids and immunosuppressive agents are fundamental in the United States.

However, no effectiveness in a long term administration aiming at maintenance of remission has been shown for specifically adrenocortical steroids, and there are many problems such as their side effects, steroid dependency and resistance. Furthermore, with respect to mesalazine and salazosulfapyridine that are 5-ASA formulations, indication for Crohn's disease is allowed only in Japan and is not allowed in the United States. It has been actually reported that mesalazine that is a 5-ASA formulation is ineffective for Crohn's disease. Despite of this fact, the formulation is used frequently by off-label use in the United States; under the actual circumstance, the formulation is reluctantly used since there is no safe oral agent. On the other hand, immunosuppressive agents such as azathioprine which have been accepted and used in Japan and the United States show a certain effect; however, side effects such as leukopenia and occurrence of lymphoma have been reported, and thus they cannot be considered to be safe drugs. Therefore, under the present circumstance, there is still no sufficient oral therapeutic drug for Crohn's disease in view of effectiveness and safeness.

Organic acid polymers, specifically 3-oxygermylpropionic acid polymers, has been studied with respect to a number of pharmaceutical use. For example, uses for hypertensive cardiovascular diseases (Jpn. Pat. Appln. Laid-open Publication No. 55-167222), an action of enhancing the production of interferons (Jpn. Pat. Appln. Laid-open Publication No. 2-134818), an action of inhibiting a Maillard reaction (Jpn. Pat. Appln. Laid-open Publication No. 8-059485), an action of enhancing the production of IL-10 (Jpn. Pat. Appln. Laid-open Publication No. 11-049683), uses for arteriosclerotic diseases (Jpn. Pat. Appln. Laid-open Publication No. 2000-229856), an action of antagonizing MCP-1 (Jpn. Pat. Appln. Laid-open Publication No. 2000-136139), use for Type II diabetic nephropathy (Jpn. Pat. Appln. Laid-open Publication No. 2003-81843) and the like have been reported.

Many supposed pharmaceutical uses of organic acid polymers are listed in the prior art documents, and as one of them, an action of healing and repairing ulcers in the gastrointestinal system, an action of controlling the function of the large intestine (Jpn. Pat. Appln. Laid-open Publication No. 54-115324, Jpn. Pat. Appln. Laid-open Publication No. 54-116100), uses for ulcers in the stomach, duodenum and large intestine (Jpn. Pat. Appln. Laid-open Publication No. 52-51327) and the like are described. As mentioned above, many pharmaceutical uses have been reported on organic acid polymers, but use for Crohn's disease has not been reported.
Patent Literature 1: Jpn. Pat. Appln. Laid-open Publication No. 55-167222
Patent Literature 2: Jpn. Pat. Appln. Laid-open Publication No. 2-134818
Patent Literature 3: Jpn. Pat. Appln. Laid-open Publication No. 8-059485
Patent Literature 4: Jpn. Pat. Appln. Laid-open Publication No. 11-049683
Patent Literature 5: Jpn. Pat. Appln. Laid-open Publication No. 2000-229856
Patent Literature 6: Jpn. Pat. Appln. Laid-open Publication No. 2003-81843
Patent Literature 7: Jpn. Pat. Appln. Laid-open Publication No. 2000-136139
Patent Literature 8: Jpn. Pat. Appln. Laid-open Publication No. 54-115324
Patent Literature 9: Jpn. Pat. Appln. Laid-open Publication No. 54-116100
Patent Literature 10: Jpn. Pat. Appln. Laid-open Publication No. 52-51327

### DISCLOSURE OF THE PRESENT INVENTION

### Problem to be Solved by the Invention

The present invention aims at providing an agent for treating Crohn's disease.

### Means for Solving the Problem

The present inventors have evaluated the medicinal effect and pharmacology of an organic acid polymer, specifically a 3-oxygermylpropionic acid polymer, in a mouse 2,4,6-trinitrobenzenesulfonic acid (TNBS)-induced Crohn's disease model. A mouse TNBS-induced Crohn's disease model is a general experimental animal model that is said to be similar to human Crohn's disease. As the result thereof, they have found that the compound has significant medicinal and pharmacological effects, and completed the present invention.

Namely, the present invention is an agent for preventing or treating Crohn's disease, comprising an organic acid polymer represented by the following formula:

[(O_{1/2})₃Ge-A-CO₂H]ₙ

wherein n is 100-1,000, and A is a lower alkyl group.

In the formula, A is preferably a C1-C3 lower alkyl group. As such organic acid polymer, a 3-oxygermylpropionic acid polymer with an ethylene group for A is preferable.

In the 3-oxygermylpropionic acid polymer, the degree of polymerization n is preferably 200-900. Such 3-oxygermylpropionic acid polymer can be specified by the following physical properties. Namely, the 3-oxygermylpropionic acid polymer that has the following features in powder X-ray diffraction spectrum: a large diffraction peak around 6.5° and relatively large diffraction peaks around 11.6°, 13.8°, 18.4°, 21.2°, and 22.4°, respectively. The 3-oxygermylpropionic acid polymer that has the following features of absorption in IR spectrum: large absorption bands around 800 cm⁻¹, 900 cm⁻¹, and 1,700 cm⁻¹, respectively, and relatively large absorption bands around 560 cm⁻¹, 705 cm⁻¹, 760 cm⁻¹, 780 cm⁻¹, 1,250 cm⁻¹, 1,350 cm⁻¹, and 1,400 cm⁻¹, respectively, the absorption band around 1,400 cm⁻¹ being a doublet. The 3-oxygermylpropionic acid polymer that has the following features of chart in DSC: a peak initiation point around 237° C., a peak top around 256° C., a peak end point around 276° C. and an amount of heat AH of approximately 59 mcal/mg. The 3-oxygermylpropionic acid polymer that has a molecular formula: (C₃H₅GeO_{3.5})ₙ, a weight average degree of polymerization: n=548±337, and a weight average molecular weight: average value±standard error=9.29×10⁴±5.72×10⁴.

### Advantageous Effects of the Invention

The organic acid polymer of the present invention is effective as an agent for treating Crohn's disease.

### BRIEF DESCRIPTION OF DRAWINGS

FIG. 1 shows a powder X-ray diffraction spectrum of propagermanium.

FIG. 2 shows an IR spectrum of propagermanium.

FIG. 3 shows DSC of propagermanium.

FIG. 4 shows pathological scores by an administration of propagermanium, salazosulfapyridine and prednisolone in mouse TNBS-induced Crohn's disease models (the effects on the first day of TNBS-initiation).

FIG. 5 shows pathological scores by the administration of propagermanium, salazosulfapyridine and prednisolone in mouse TNBS-induced Crohn's disease models (the effects on the second day of TNBS-initiation).

FIG. 6 shows pathological scores by the administration of propagermanium, salazosulfapyridine and prednisolone in mouse TNBS-induced Crohn's disease models (the effects on the third day of TNBS-initiation).

FIG. 7 shows intestinal lesion scores by the administration of propagermanium, salazosulfapyridine and prednisolone in mouse TNBS-induced Crohn's disease models.

### BEST MODE FOR CARRYING OUT THE INVENTION

The organic acid polymer used in the present invention is a known compound, and is represented by the following chemical formula:

[(O_{1/2})₃Ge-A-CO₂H]ₙ

wherein n is 100-1,000, and A is a lower alkyl group.

Here, the lower alkyl group for A is preferably a C1-C3 lower alkyl group. Specifically, a 3-oxygermylpropionic acid polymer with an ethylene group for A is preferable. In the 3-oxygermylpropionic acid polymer, propagermanium having a degree of polymerization n of 200-900 is well known. The steric structure thereof is presumed to be an 8-membered ring structure represented by the following formula:

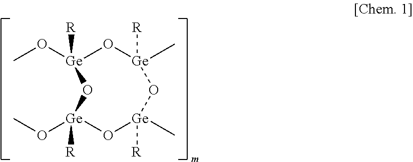

Wherein R represents —CH₂CH₂COOH, and m is a weight average degree of polymerization obtained by converting from the weight average molecular weight of a propagermanium propyl ester and is 137±84 (average value±standard error 3τ). The minimum structural unit: (O_{1/2})₃GeCH₂CH₂COOH, experimental formula: C₆H₁₀Ge₂O₇.

The propagermanium can be produced according to the method described in Jpn. Pat. Appln. Laid-open Publication No. 2003-81843 or the like. Furthermore, the literature and the like describe that the propagermanium has the physical properties shown in the following Tables 1 and 2. Table 1 shows the result of the molecular weight measurement by a light scattering method, and Table 2 shows a lattice constant obtained by a powder X-ray analysis.

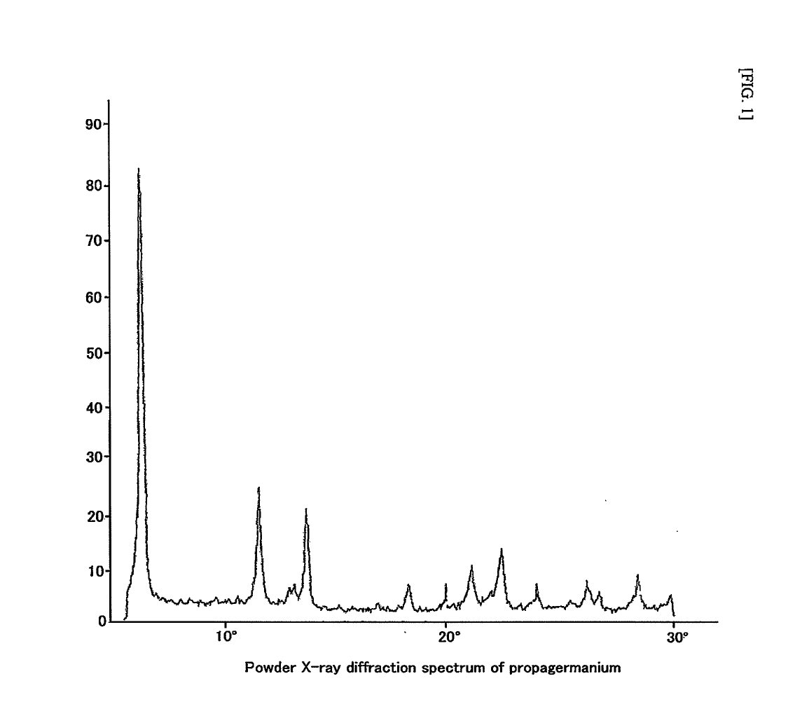

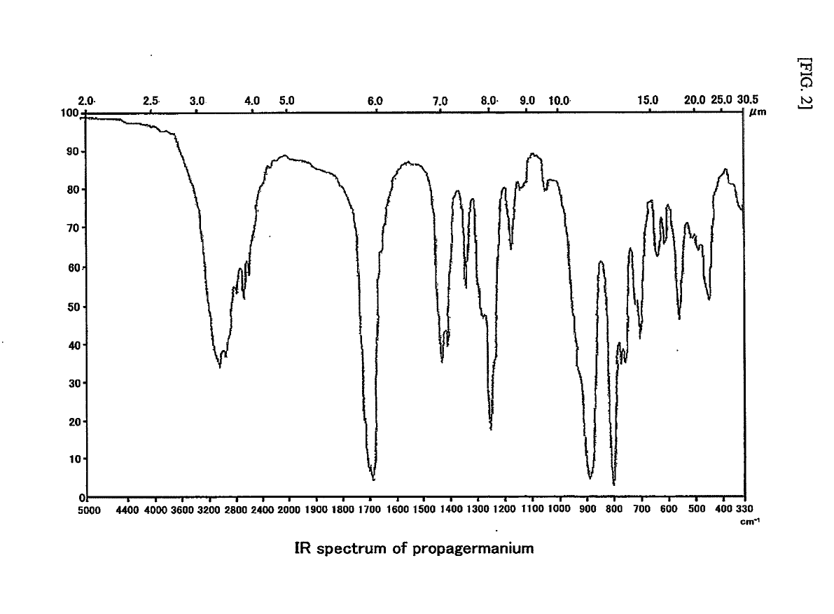

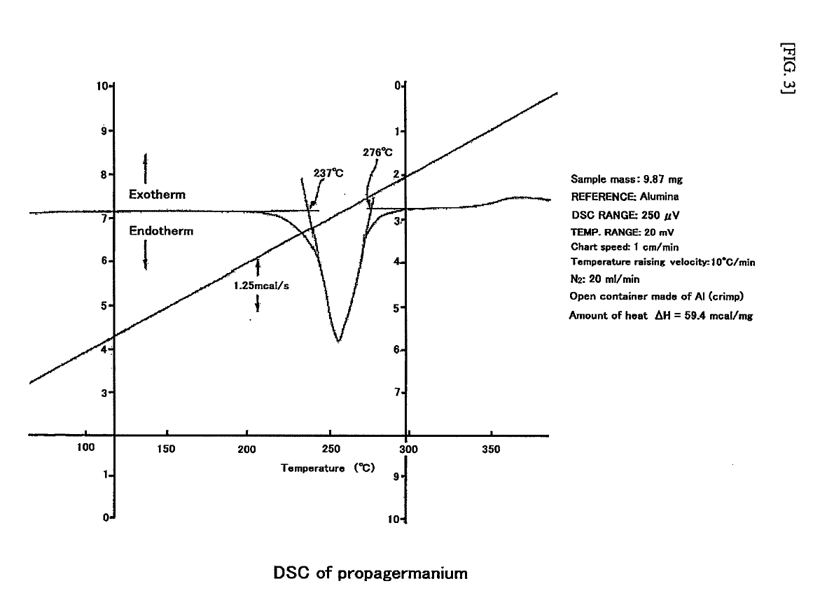

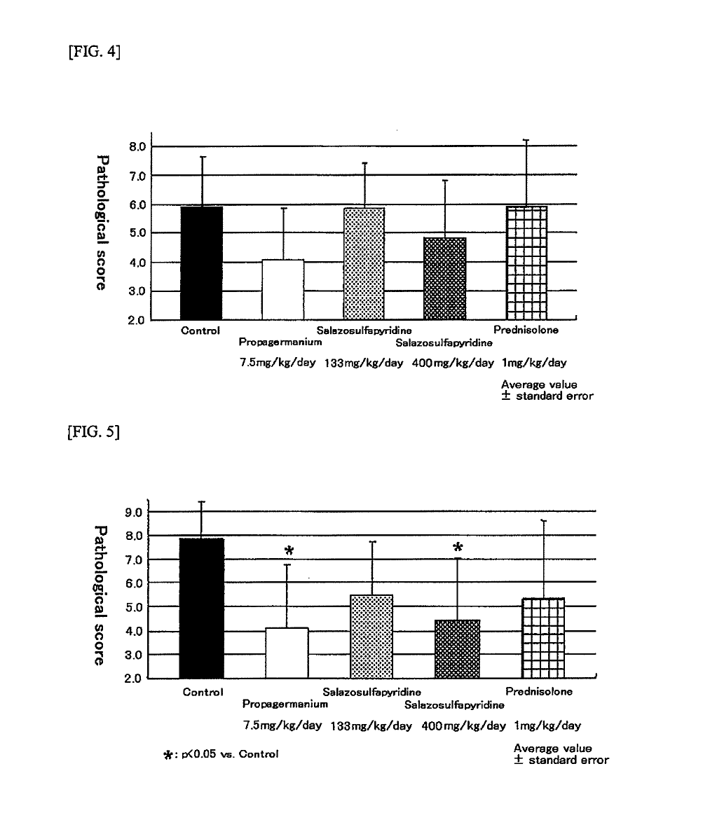

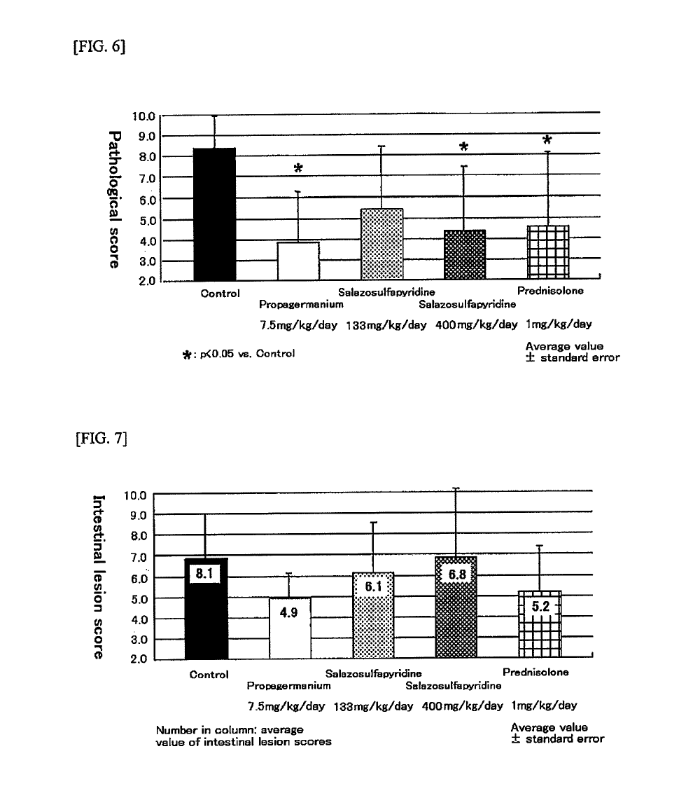

The propagermanium can be specified as a compound having the following physical properties according to FIGS. 1 to 3 (cited from Jpn. Pat. Appln. Laid-open Publication No. 54-115324). It has the following features in powder X-ray diffraction spectrum: a large diffraction peak around 6.5° and relatively large diffraction peaks around 11.6°, 13.8°, 18.4°, 21.2°, and 22.4°, respectively. It has the following features of absorption in IR spectrum: large absorption bands around 800 cm⁻¹, 900 cm⁻¹, and 1,700 cm⁻¹, respectively, and relatively large absorption bands around 560 cm⁻¹, 705 cm⁻¹, 760 cm⁻¹, 780 cm⁻¹, 1,250 cm⁻¹, 1,350 cm⁻¹, and 1,400 cm⁻¹, respectively, the absorption band around 1,400 cm⁻¹ being a doublet. It has the following features of chart in DSC: a peak initiation point around 237° C., a peak top around 256° C., a peak end point around 276° C. and an amount of heat AH of approximately 59 mcal/mg.

TABLE 1
Weight average molecular weight of propagermanium

| | |
|---|---|
| Propagermanium | Propagermanium |
| propyl ester | (corresponding value) |

Weight average molecular weight (Mw)

| | | |
|---|---|---|
| Average value ( X̅) | 1.16 × 10⁵ | 9.29 × 10⁴ |
| Standard error (3s) | ±0.71 × 10⁵ | ±5.72 × 10⁴ |
| Molecular formula* | (C₆H₁₁GeO_{3.5})ₙ | (C₃H₅GeO_{3.5})ₙ |
| Weight average | 548 ± 337 | 548 ± 337 |
| degree of polymerization (n)* | | |

*is a value in the case the minimum structural unit of propagermanium is (O_{1/2})₃GeCH₂CH₂COOH.

TABLE 2
Lattice constant of propagermanium

| | |
|---|---|
| Chemical formula*¹ | (C₃H₅GeO_{3.5})ₙ |
| Formula weight*¹ | 169.66 |
| Crystal class | monoclinic |
| Space group | — |
| Unit cell parameters | |
| a (Å) | 13.35*¹ |
| b (Å) | 5.03*¹ |
| c (Å) | 7.55*¹ |
| β (deg.) | 94.3*¹ |
| vol (Å³) | 505.4*² |
| z | 4*³ |
| density (gcm⁻³) | 2.23*⁴ |

*¹The repeating unit of propagermanium is represented as (O_{1/2})₃GeCH₂CH₂COOH.
*²Calculated from the lattice constant
*³Calculated from the lattice constant and measured density
*⁴Measured by a flotation method

In the case the 3-oxygermylpropionic acid polymer, particularly the propagermanium used in the present invention is actually administered to a human, it is preferably used as a composition that is prepared so as to contain excipients in the range of 0.005 part by mass to 50 parts by mass with respect to the compound in the range of 0.005 part by mass to 5 parts by mass. As the excipients, sugars such as lactose, sucrose and dextrans, cellulose-based polymeric substances such as hydroxypropyl cellulose, and natural polymer substances such as albumin are used. Furthermore, although the compound is generally used as an oral formulation, it can also be utilized as a suppository, a nasal cavity formulation, an injection formulation or the like. The dose in the case the compound is administered to a human is within the range of 1 mg to 1,500 mg per day depending on the dosage form, the age of the patient, and the like, and is preferable within the range of 10 mg to 120 mg per day for oral administration to an adult human having a body weight of 50 kg. Furthermore, the formulation of the compound can be conducted according to the description of the formulation example of Jpn. Pat. Appln. Laid-open Publication No. 2003-81843 or the like.

Crohn's disease is an idiopathic chronic disease of unknown cause by which non-specific lesions appear incontinuously on the whole area of the gastrointestinal tract mainly from the oral cavity to the anus.

The clinical symptoms thereof are abdominal pain, general malaise, diarrhea, bloody stool, fever, weight-loss, anemia, ileus, abdominal mass, nausea, vomiting, peritonitis, and the like. Crohn's disease simultaneously causes nutritional disorder, as well as various gastrointestinal and extraintestinal symptoms, for example, severe symptoms such as intestinal stenosis, ileus, internal fistula, external fistula, intestinal perforation, abdominal mass and massive bleeding. Furthermore, in some cases, the disease is accompanied by extraintestinal complications such as arthritis, iritis, pyoderma gangrenosum and erythema nodosum.

As the treatment method therefor, a nutrition therapy or medicinal therapy is mainly adopted, but a radical operation therapy cannot be expected.

### EXAMPLES

The preparation example of the substance of the present invention will be shown below.

### Preparation Example 1

Germanium dioxide and 50% hypophosphorous acid (1.1-fold mol) were reacted in the presence of concentrated hydrochloric acid (5.0-fold mol) at 60 to 80° C. for 4 hours, concentrated hydrochloric acid (5.0-fold mol) was added thereto, and acrylic acid (1.1-fold mol) was added dropwise thereto at 40° C. or less. The precipitated crystal was collected by filtration and washed with concentrated hydrochloric acid to obtain 3-trichlorogermylpropionic acid (yield 98%). The obtained 3-trichlorogermylpropionic acid was then dissolved in acetone (17-fold mol) and filtered, thereafter water (70-fold mol) was added dropwise thereto at 0° C. under stirring, and the mixture was further stirred for 6 hours and stood still for 16 hours. The precipitated crystal was collected by filtration and washed with acetone to obtain a 3-oxygermylpropionic acid polymer (yield 92%).

The obtained 3-oxygermylpropionic acid polymer, i.e., propagermanium, was further induced into a propyl ester form, and thereafter the molecular weight was measured by a light scattering method, and the lattice constant was measured by a powder X-ray analysis method. The results were as shown in Table 1 and Table 2, respectively.

Next, the example of the pharmacological test on the medicinal effect of the propagermanium will be shown. In the pharmacological test on the medicinal effect, the propagermanium was evaluated in a mouse TNBS-induced Crohn's disease model.

Since weight-loss, diarrheal bloody stool and intestinal lesions occur in the model, the model is a general pathological animal model that is said to be similar to human Crohn's disease.

As two kinds of positive control agents, prednisolone and salazosulfapyridine were used. Prednisolone is one of major drugs for the treatment of Crohn's disease as an adrenocortical steroid drug. Since salazosulfapyridine is a prodrug form of a 5-ASA formulation and is converted to 5-ASA in the large intestine, it is a more preferable positive control drug than 5-ASA formulations in view of the drug concentration in the intestinal tract that is a site of action in animal tests.

In this pharmacological test on the medicinal effect, the propagermanium produced in the above-mentioned Preparation Example 1 was used.

### Example 1 of Pharmacological Test on Medicinal Effect

### 1. EXPERIMENTAL METHOD

Seven-week-old male BALB/c Cr Slc mice were purchased from Japan SLC, Inc. After 5 days of breeding for taming, the mice were divided into 5 groups so as to eliminate the difference between body weights, and subjected to an experiment.

The hair on 2 square-cm of the abdomen of the mouse was shaved, and 1% TNBS was applied to the skin. At 7 days after the application of TNBS, the mouse was fixed under anesthesia by halothane inhalation, and 100 μL of 2.0% TNBS was injected from the rectum to initiate intestinal lesions in Crohn's disease.

In the propagermanium-administered group, 0.015% of the propagermanium was mixed with a standard feedstuff for mice CRF-1 (manufactured by Oriental Yeast Co., Ltd.) and administered by ad libitum feeding from immediately after the injection of TNBS to 3 days after the initiation. The average dose during the period of the administration of the propagermanium was calculated to be about 7.5 mg/kg/day as a dose of the propagermanium per day, from the body weight and food consumption.

In the prednisolone-administered group as a positive control, prednisolone was administered once a day as 10 mL/kg of 0.5% CMC suspension using an oral canula for mice from immediately after the injection of TNBS to 3 days after the initiation. The dose was set to be 1 mg/kg/day that is the maximum amount of a clinical general dose (30 to 40 mg/day/person).

In the salazosulfapyridine-administered group as a positive control, salazosulfapyridine was administered twice a day for 3 days as 10 mL/kg of 0.5% CMC suspension using an oral canula for mice from immediately after the injection of TNBS to 3 days after the initiation. Two dose groups: a low dose group (133 mg/kg/day) that is the maximum amount in view of a clinical general dose and a high dose group (400 mg/kg/day) that is effective in rat TNBS models, were set.

### 2. EVALUATION ITEMS

### (1) Pathological Score

At 1, 2 and 3 days after the TNBS initiation, the pathological score (the total of the scores of weight-loss, diarrhea/loose stool and occult blood/hemorrhage) was evaluated according to the following criteria.

(A) Weight-loss score (when the body weight before the TNBS initiation is considered to be 100%)
0: No decrease
1: Decreased by within 5%
2: Decreased by 5-10%
3: Decreased by 10-20%
4: Decreased by more than 20%

(B) Diarrhea/loose stool score
0: Normal. A solid form is maintained.
2: Semi-solid loose stool. Wet, and readily collapses when being touched.
4: Diarrhea, with flowability.

(C) Occult blood/hemorrhage score
0: Normal. No blood is incorporated in stool.
2: Occult blood. Blood is incorporated in stool. Perianal is slightly dirty.
4: Bloody stool. Blood is incorporated in stool. Perianal is dirty with blood.

### (2) Intestinal Lesion Score

At 3 days after the TNBS initiation, Evans Blue was administered intravenously. At 30 minutes after the administration, the mouse was sacrificed by cervical dislocation, from the colon to the anus were excised and the inner cavity was washed with physiological saline, the lumen was opened, and the degree of the lesions was scored. The intestinal lesion score was evaluated by observing the colon by using a stereomicroscope according to the following criteria.
0: No damage.
1: Hyperemia
2: Hyperemia and thickening of gut walls. No ulcer.
3: Ulcer is present, but is not thickened.
4: Plural lesion sites are present.
5: Widespread lesion sites (1 cm or more in total)
6: Widespread lesion sites (1.5 cm or more in total)
7: Widespread lesion sites (2 cm or more in total)
8: Widespread lesion sites (2.5 cm or more in total)
9: Widespread lesion sites (3 cm or more in total)
10: Widespread lesion sites (3.5 cm or more in total)

### 3. RESULTS AND DISCUSSION

### (1) Effect on Pathological Score

In the 7.5 mg/kg of propagermanium-administered group, a significant effect of improving the pathological score was confirmed at 2 days and 3 days after the initiation. On the other hand, a significant improving effect was not observed in the 133 mg/kg of salazosulfapyridine-administered group. In the 400 mg/kg of salazosulfapyridine-administered group, an improving effect was found at 2 days and 3 days after the initiation, but the effect was weaker than that of the propagermanium group.

On the other hand, in the group of 1 mg/kg of prednisolone, whose reliability is the highest in view of an effect of inducing remission against Crohn's disease, was administered, the effect was not observed at 2 days after the initiation and the effect was observed only at 3 days after the initiation, but the improving effect was weaker than that of the 7.5 mg/kg of the propagermanium-administered group.

### (2) Intestinal Lesion Score

The effect of improving the intestinal lesion score was the most excellent in the 7.5 mg/kg of propagermanium-administered group, as compared to the 133 mg/kg of salazosulfapyridine-administered group, the 400 mg/kg of salazosulfapyridine-administered group and the 1 mg/kg of prednisolone-administered group.

Namely, as compared to the control group (score 8.1) on lesion, the score was decreased to 4.9 in the propagermanium group; the decreasing effect was weak as 6.1 in the salazosulfapyridine 133 mg/kg group and 6.8 in the salazosulfapyridine 400 mg/kg group; the decreasing effect was 5.2 in the prednisolone 1 mg/kg group; and thus the score was the most excellent in the propagermanium group.

From these results, since the propagermanium showed a more excellent effect than those of prednisolone and salazosulfapyridine, effects on both induction of remission and maintenance of remission state in Crohn's disease can be expected.

1-8. (canceled)

9. A method for preventing or treating Crohn's disease in a mammal, comprising a step for administering to the mammal an effective amount of an organic acid polymer represented by the following formula:
[(O_{1/2})₃Ge-A-CO₂]ₙ
wherein n is 100-1,000, and A is a lower alkyl group.

10. The method according to claim 9, wherein A is a C1-C3 lower alkyl group.

11. The method according to claim 9, wherein the organic acid polymer is a 3-oxygermylpropionic acid polymer with an ethylene group for A.

12. The method according to claim 11, wherein the degree of polymerization n of the 3-oxygermylpropionic acid polymer is 200-900.

13. The method according to claim 11, wherein the 3-oxygermylpropionic acid polymer has the following features in powder X-ray diffraction spectrum:
a large diffraction peak: around 6.5°,
relatively large diffraction peaks: around 11.6°, 13.8°, 18.4°, 21.2°, and 22.4°, respectively.

14. The method according to claim 11, wherein the 3-oxygermylpropionic acid polymer has the following features of absorption in IR spectrum:
large absorption bands: around 800 cm⁻¹, 900 cm⁻¹, and 1,700 cm⁻¹, respectively,
relatively large absorption bands: around 560 cm⁻¹, 705 cm⁻¹, 760 cm⁻¹, 780 cm⁻¹, 1,250 cm⁻¹, 1,350 cm⁻¹, and 1,400 cm⁻¹, respectively, the absorption band around 1,400 cm⁻¹ being a doublet.

15. The method according to claim 11, wherein the 3-oxygermylpropionic acid polymer has the following features of chart in DSC:
peak initiation point: around 237° C.,
peak top: around 256° C.,
peak end point: around 276° C.,
amount of heat ΔH=approximately 59 mcal/mg.

16. The method according to claim 11, wherein the 3-oxygermylpropionic acid polymer is a compound that has:
a molecular formula: (C₃H₅GeO_{3.5})ₙ,
a weight average degree of polymerization: n=548±337, and
a weight average molecular weight: average value±standard error=9.29×10⁴±5.72×10⁴.
